(19) United States
(12) Patent Application Publication
(10) Pub. No.: US 2016/0280691 A1
(43) Pub. Date: Sep. 29, 2016

(54) TRIAZOLE INTERMEDIATES USEFUL IN THE SYNTHESIS OF PROTECTED N-ALKYLTRIAZOLECARBALDEHYDES

(71) Applicant: BIOMARIN PHARMACEUTICAL INC., Novato, CA (US)

(72) Inventor: Mark HENDERSON, Larkspur, CA (US)

(21) Appl. No.: 15/035,193

(22) PCT Filed: Nov. 6, 2014
(86) PCT No.: PCT/US2014/064273

§ 371 Date: May 6, 2016

Related U.S. Application Data
(60) Provisional application No. 61/901,300, filed on Nov. 7, 2013.

(51) Int. Cl. C07D 405/06; C07D 249/08

(52) U.S. Cl. CPC C07D 405/06; C07D 249/08

(57) ABSTRACT

Described herein are compounds and methods of making such compounds useful in the synthesis of protected N-alkyl-triazolecarbaldehydes.

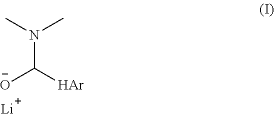

### CROSS REFERENCE TO RELATED APPLICATIONS

The present application claims the benefit of priority of U.S. Provisional Application No. 61/901,300, filed Nov. 7, 2013, the content of which is hereby incorporated by reference in its entirety.

### FIELD

Described herein are protected-N-alkyl-triazolecarbaldehydes and methods of making such compounds.

### BACKGROUND

There are several methods of making N-alkyl-triazolecarbaldehydes known in the art. U.S.20090318436 discloses the synthesis of 2-methyl-2H-1,2,4-triazole-3-carbaladehyde via treatment of 2-methyl-2H-1,2,4-triazole with i-PrMgCl in THF which is followed by addition of DMF and extraction with DCM. WO2004024691 and WO2008135826 disclose the synthesis of N-alkyl-triazolecarbaldehydes by treating the N-alkyl-triazole with n-butyllithium, followed by treatment with DMF and extraction or chromatography using DCM. Ivanova et al. (Synthesis 2006(1): 156-160) and WO2005080356 disclose the synthesis of N-alkyl-triazolecarbaldehydes by treating hydroxymethyl-N-alkyl-triazoles with MnO₂ in a solvent such as THF or DCM. WO2003002567 discloses the synthesis of N-alkyl-1,3,4-triazolecarbaldehydes by treating diethyoxyethyl-N-alkyl-1,3,4-triazole with H₂SO₄ at elevated temperatures (75-80° C.).

Current syntheses of N-alkyl-triazolecarbaldehydes have several drawbacks. For example, elevated temperatures or very cold temperatures (−60° C. for example) are required. DCM, used in the workup or purification steps of some of the prior art syntheses, is associated with liver toxicity and is an environmentally undesirable solvent. In addition, n-butyllithium is pyrophoric and thus dangerous to handle. In some cases, syntheses are low-yielding and cannot be performed on a large scale and thus are inefficient. Finally, regardless of how the N-alkyltrizolecarbaldehydes are made, they are not stable in solution and pose an explosion risk.

The present methods gave a surprising result. A base screening (KOtBu, NaOtBu, LiOtBu, and NaHMDS) was performed to replace the pyrophoric n-BuLi where after complete addition of the base, DMF was added. In all cases the product was soluble and thus workup and purification led to low yields. However, when LiHDMS was used as the base but was added after the DMF, the product was isolated as a non-hygroscopic and stable solid. This solid can be used directly where the unstable N-alkyltrizolecarbaldehyde would have been used.

In light of the drawbacks of the current methods of making and using N-alkyl-triazolecarbaldehydes, the methods and related intermediates disclosed herein are needed.

### BRIEF DESCRIPTION OF THE DRAWINGS

FIGS. 1a. and 1b., respectively, depict the ¹H NMR (CD₃OD) and ¹³C NMR (CD₃OD) for:

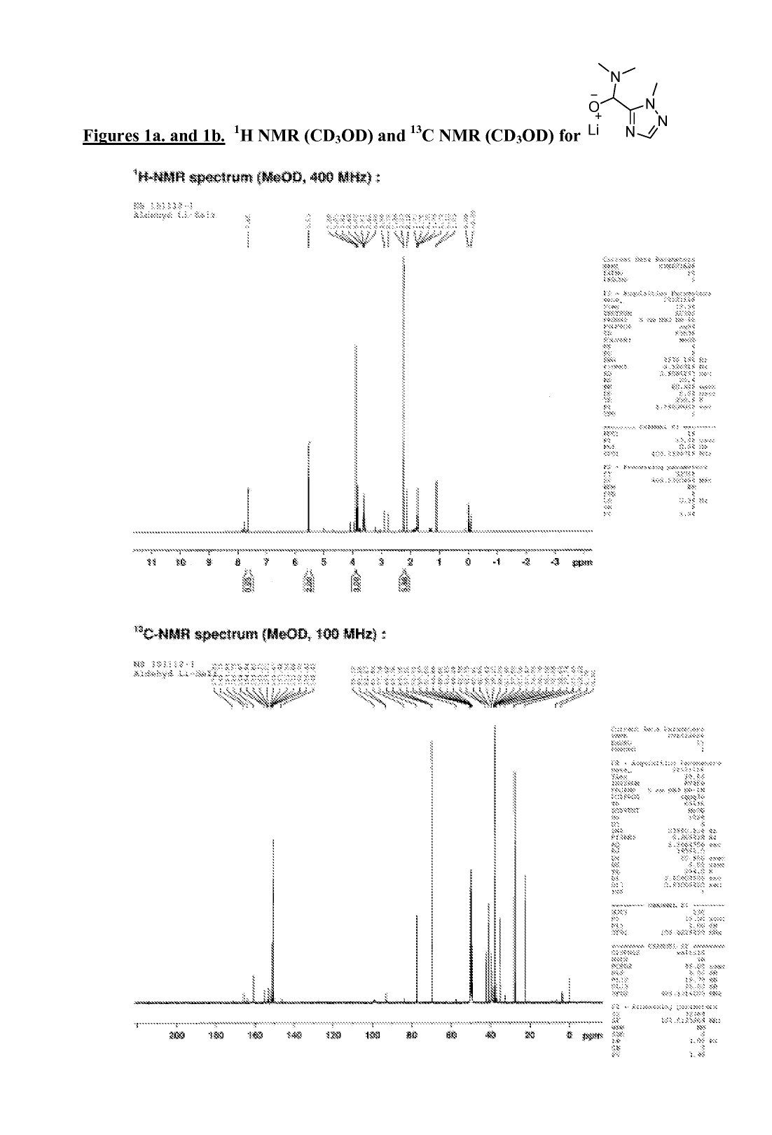

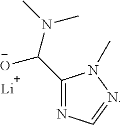

FIGS. 2a. and 2b., respectively, depict the ¹H-¹H COSY(CD₃OD) and ¹³C-¹H HSQC (CD₃OD) NMR for:

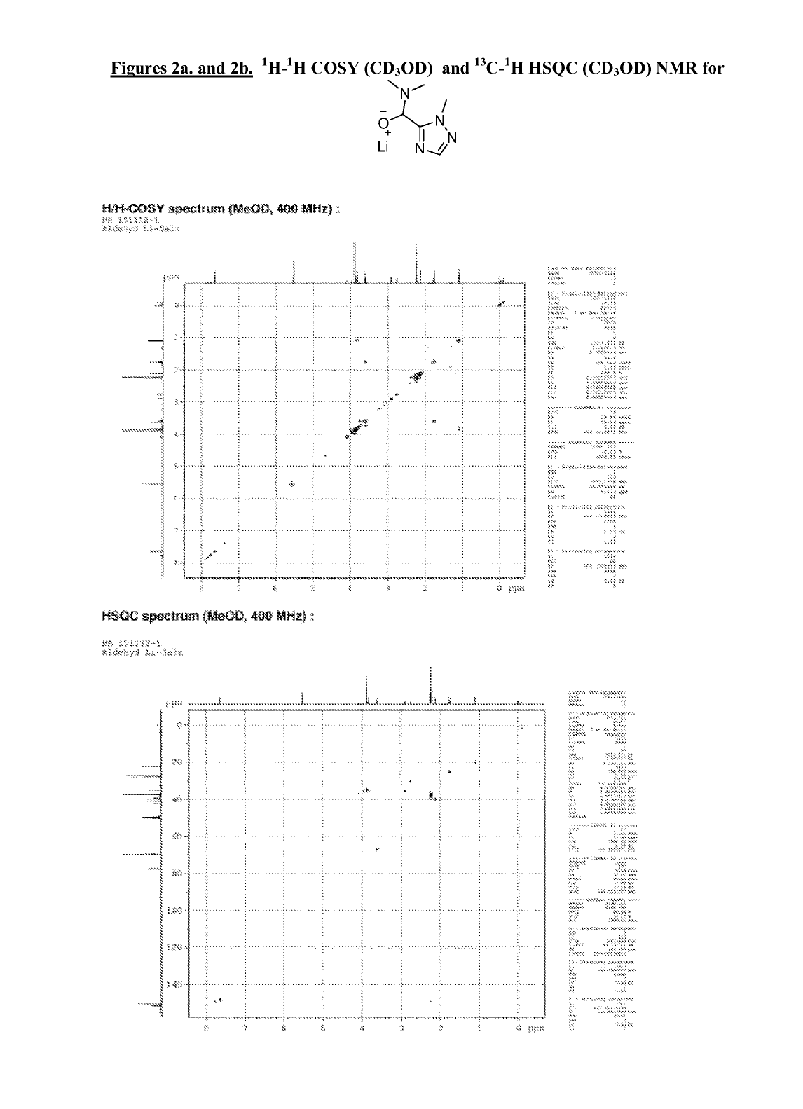

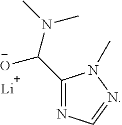

FIGS. 3a. and 3b., respectively, depict the ¹H DEPT (CD₃OD) and ¹³C “CH only” (CD₃OD) NMR for:

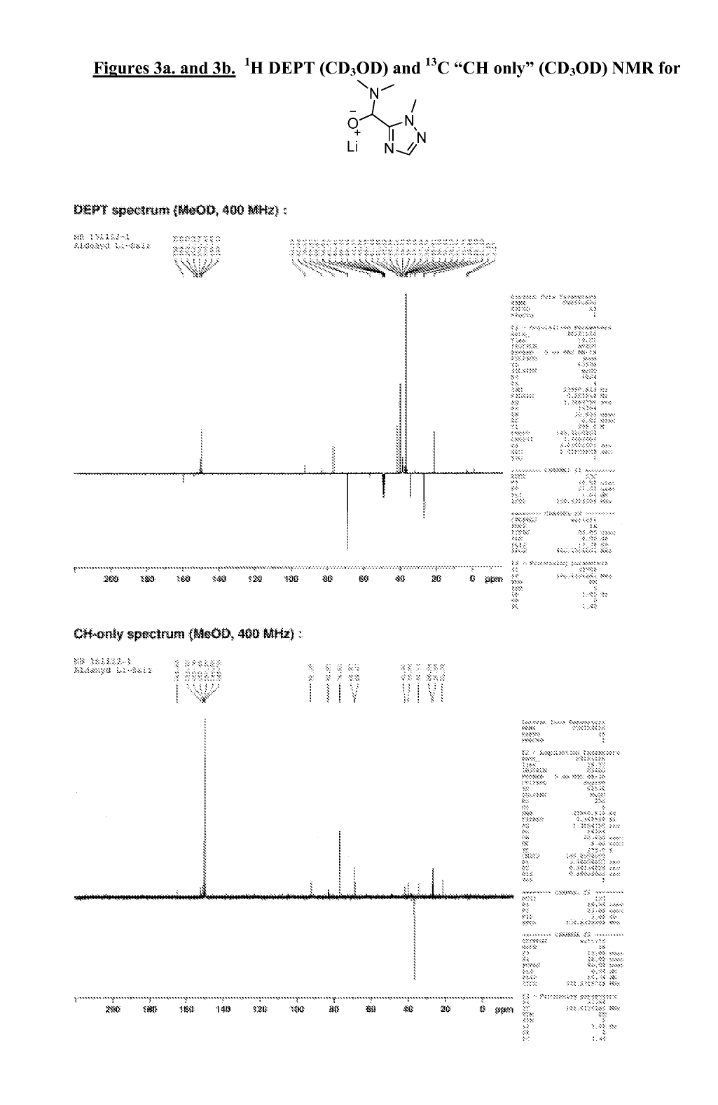

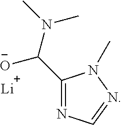

FIG. 4. depicts the IR spectrum, run as a KBr disk, for:

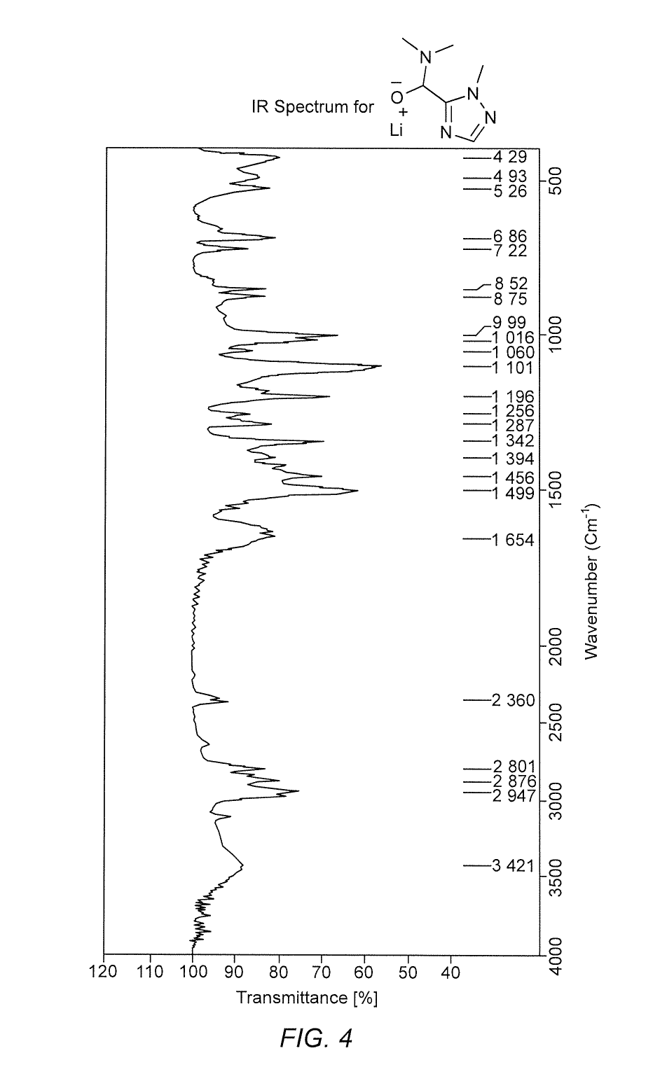

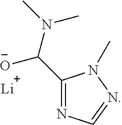

FIG. 5. depicts the DSC, run at 2° C./minute from 50 to 300° C. on a solid sample, for

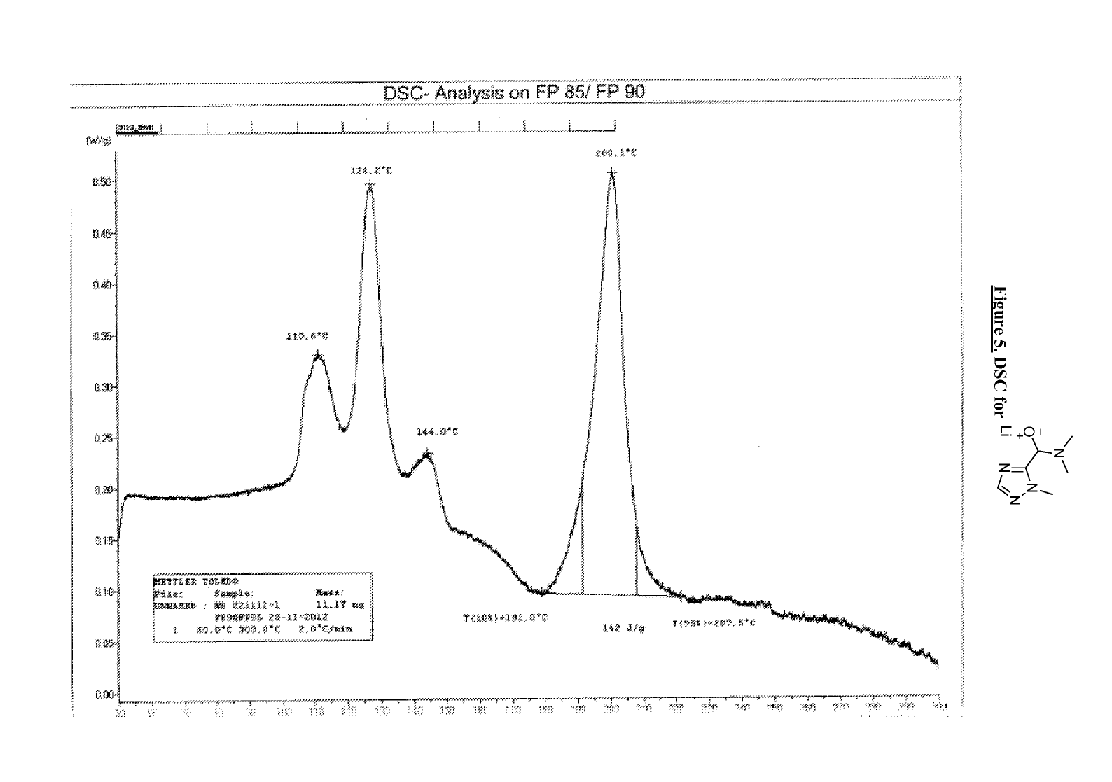

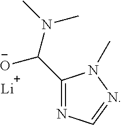

### SUMMARY OF THE INVENTION

In one aspect, provided herein is a compound of Formula I:

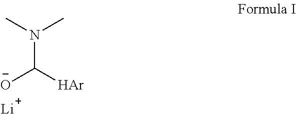

where HAr is N-alkyl-1,2,4-triazolyl, N-alkyl-1,3,4-triazolyl, or N-alkyl-1,2,3-triazolyl.

In a further aspect, provided herein is a method of making a compound of Formula I,

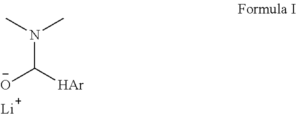

comprising:
a) treating an intermediate of formula HAr-H, or a salt thereof, where HAr is as defined in the Summary of the Invention or as in any of the embodiments described herein; with DMF followed by a lithium base to yield a compound of Formula I; and
b) optionally further comprising treating the compound of Formula I and 6-fluoro-4-nitroisobenzofuran-1(3H)-one with acetic acid or acetic anhydride in the presence of water and a base to yield a compound of Formula II:

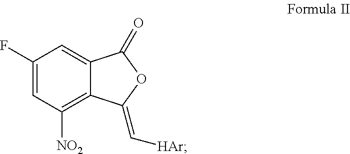

where HAr is as defined in the Summary of the Invention or as in any of the embodiments described herein.

### DETAILED DESCRIPTION

### Abbreviations

| Abbreviation | Meaning |
|---|---|
| COSY | correlation spectroscopy |
| DCM | dichloromethane |
| DEPT | distortionless enhancement by polarization transfer |
| | (spectroscopy) |
| DIPEA | N,N-diisopropyl-N-ethylamine |
| DSC | differential scanning calorimetry |
| DMF | N,N-dimethylformamide |
| equiv | equivalent |
| g | gram |
| HSQC | heteronuclear single quantum correlation (spectroscopy) |
| IR | infrared |
| KOtBu | potassium tert-butoxide |
| LDA | lithium diisopropylamide |
| LiHMDS | lithium bis(trimethylsilyl)amide |
| LiOtBu | lithium tert-butoxide |
| MHz | megahertz |
| mL | milliliter |
| mol | mole |
| m/v | molarity of the solution/volume of the solution |
| NaHMDS | sodium bis(trimethylsilyl)amide |
| NaOtBu | sodium tert-butoxide |
| NMR | nuclear magnetic resonance (spectroscopy) |
| THF | tetrahydrofuran |

### Definitions

To facilitate understanding of the disclosure set forth herein, a number of terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

As used throughout this application and the appended claims, the following terms have the following meanings:

“About” preceding a numerical value refers to a range of values ±10% of the value specified.

“Alkyl” means a linear or cyclic, straight or branched, saturated hydrocarbon radical containing from 1-10 carbon atoms, in another example 1-6 carbon atoms. Illustrative examples include, but are not limited to, methyl, ethyl, n-propyl, iso-propyl, cyclopropyl, n-butyl, sec-butyl, iso-butyl, tert-butyl, cyclobutyl, n-pentyl, isopentyl, neopentyl, cyclopentyl, n-hexyl, 3-methylhexyl, 2,2-dimethylpentyl, 2,3-dimethylhexyl, cyclohexyl, n-heptyl, n-octyl, n-nonyl, and n-decyl.

“Stereoisomers” include (but are not limited to) geometric isomers, enantiomers, diastereomers, and mixtures of geometric isomers, enantiomers or diastereomers. In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral auxiliary, separation of the resulting mixture of diastereomers by recrystallization or chromatography and liberation of the optically pure product from the auxiliary, or (2) direct separation of the mixture of optical enantiomers on chiral chromatographic column.

### Embodiments

The following paragraphs present a number of embodiments of the compounds disclosed herein. In each instance the embodiment includes both the recited compound(s) as well as a single stereoisomer or mixture of stereoisomers thereof. In some situations, the compounds exist as tautomers. All tautomers are included within the scope of the compounds presented herein.

### Compounds of Formula I and II

In some or any embodiments, the compound of Formula I and II is that where the HAr is N-alkyl-1,2,4-triazolyl. In some or any embodiments, the alkyl is C₁₋₆ alkyl. In some or any embodiments, the alkyl is methyl, ethyl, or propyl. In some or any embodiments, the alkyl is methyl. In some or any embodiments, the alkyl is ethyl. In some or any embodiments, the alkyl is propyl. In some or any embodiments, the compound of Formula I and II is that where the HAr is N-methyl-1,2,4-triazolyl, N-ethyl-1,2,4-triazolyl, N-(n-propyl)-1,2,4-triazolyl, N-(isopropyl)-1,2,4-triazolyl, N-cyclopropyl-1,2,4-triazolyl, N-(n-butyl)-1,2,4-triazolyl, N-(sec-butyl)-1,2,4-triazolyl, N-(isobutyl)-1,2,4-triazolyl, N-(tert-butyl)- 1,2,4-triazolyl, or N-cyclobutyl-1,2,4-triazolyl. In some or any embodiments, the compound of Formula I and II is that where the HAr is N-methyl-1,2,4-triazolyl or N-ethyl-1,2,4-triazolyl. In some or any embodiments, the compound of Formula I and II is that where the HAr is N-methyl-1,2,4-triazolyl.

In some or any embodiments, the compound of Formula I and II is that where the HAr is N-alkyl-1,3,4-triazole. In some or any embodiments, the alkyl is C₁₋₆ alkyl. In some or any embodiments, the alkyl is methyl, ethyl, or propyl. In some or any embodiments, the alkyl is methyl. In some or any embodiments, the alkyl is ethyl. In some or any embodiments, the alkyl is propyl. In some or any embodiments, the compound of Formula I and II is that where the HAr is N-methyl-1,3,4-triazolyl, N-ethyl-1,3,4-triazolyl, N-(n-propyl)-1,3,4-triazolyl, N-(isopropyl)-1,3,4-triazolyl, N-cyclopropyl-1,3,4-triazolyl, N-(n-butyl)-1,3,4-triazolyl, N-(sec-butyl)-1,3,4-triazolyl, N-(isobutyl)-1,3,4-triazolyl, N-(tert-butyl)- 1,3,4-triazolyl, or N-cyclobutyl-1,3,4-triazolyl. In some or any embodiments, the compound of Formula I and II is that where the HAr is N-methyl-1,3,4-triazolyl or N-ethyl-1,3,4-triazolyl. In some or any embodiments, the compound of Formula I and II is that where the HAr is N-methyl-1,3,4-triazolyl.

In some or any embodiments, the compound of Formula I and II is that where the HAr is N-alkyl-1,2,3-triazole. In some or any embodiments, the alkyl is C₁₋₆ alkyl. In some or any embodiments, the alkyl is methyl, ethyl, or propyl. In some or any embodiments, the alkyl is methyl. In some or any embodiments, the alkyl is ethyl. In some or any embodiments, the alkyl is propyl. In some or any embodiments, the compound of Formula I and II is that where the HAr is N-methyl-1,2,3-triazolyl, N-ethyl-1,2,3-triazolyl, N-(n-propyl)-1,2,3-triazolyl, N-(isopropyl)-1,2,3-triazolyl, N-cyclopropyl-1,2,3-triazolyl, N-(n-butyl)-1,2,3-triazolyl, N-(sec-butyl)-1,2,3-triazolyl, N-(isobutyl)-1,2,3-triazolyl, N-(tert-butyl)-1,2,3-triazolyl, or N-cyclobutyl-1,2,3-triazolyl. In some or any embodiments, the compound of Formula I and II is that where the HAr is N-methyl-1,2,3-triazolyl or N-ethyl-1,2,3-triazolyl. In some or any embodiments, the compound of Formula I and II is that where the HAr is N-methyl-1,2,3-triazolyl.

### Methods of Preparing Compounds of Formula I

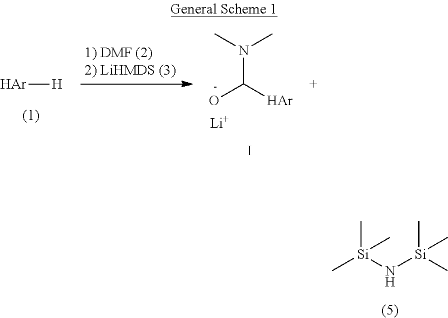

A compound of Formula I (also referred to as “Compound I”) where HAr is as defined in the Summary of the Invention or according to any of the embodiments disclosed herein can be prepared according to General Scheme 1. HAr-H (1) is treated with DMF in a first solvent, wherein the first solvent is tetrahydrofuran, 2-methyl-tetrahydrofuran, an alkyl furan (a furan substituted with 1 or 2 C₁₋₄ alkyl groups), tert-butylmethylether, cyclopentylmethylether, or dioxane. The reaction is cooled, for example, to about −5 to 0° C. and LiHMDS (3) is then added dropwise over, for example, about 60 minutes. Other lithium bases may be used, such as lithium diisopropylamide, lithium amide (LiNH₂), or lithium hydride (LiH). The reaction is stirred for about 30 minutes, for example, and the product precipitates. The precipitate can be a solvated form of Compound I, such as a Compound I-tetrahydrofuran solvate or Compound I-2-methyl-tetrahydrofuran solvate. The product is then collected by filtration and washed with a second solvent such as 2-methyl-tetrahydrofuran. Alternative second solvents include tetrahydrofuran, an alkyl furan (a furan substituted with 1 or 2 C₁₋₄ alkyl groups), tert-butylmethylether, cyclopentylmethylether, or dioxane. The product, Compound I, can then be obtained by drying, for example under vacuum and optionally with heating, for example to about 60° C.

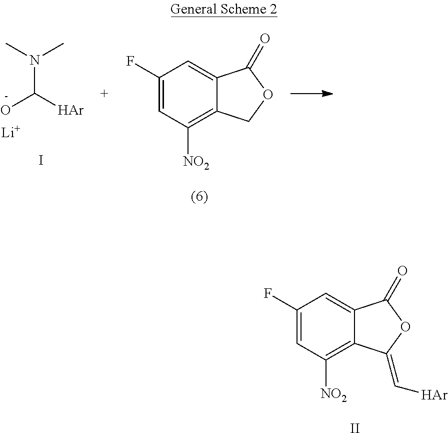

Compound I can then be used directly in a subsequent reaction instead of the corresponding aldehyde, e.g. see General Scheme 2. For example, to a mixture of Compound I and an optionally substituted isobenzofuranone, such as 6-fluoro-4-nitroisobenzofuran-1(3H)-one (6), in a solvent such as 2-methyl-tetrahydrofuran is added acetic anhydride dropwise. Other solvents include tetrahydrofuran, an alkyl furan (a furan substituted with 1 or 2 C₁₋₄ alkyl groups), tert-butylmethylether, cyclopentylmethylether, or dioxane. The mixture is heated to about 45° C., a base is added, and the reaction is allowed to proceed for about 5 hours. The mixture can then be cooled to about 20° C. and water is added dropwise. The mixture is stirred for about 30 minutes. The product precipitates, is collected by filtration and is washed with a solvent such as 2-methyl-tetrahydrofuran, followed by water and then a solvent such as methanol. The precipitate can then be dried under vacuum with heating at about 60° C. to yield Compound II.

In some or any embodiments, the method of making a compound of Formula I,

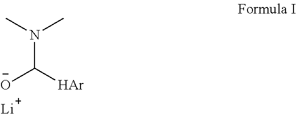

comprises:
a) treating an intermediate of formula HAr-H, or a salt thereof, where HAr is as defined in the Summary of the Invention or as in any of the embodiments described herein; with DMF followed by LiHMDS to yield a compound of Formula I; and
b) optionally further comprising treating the compound of Formula I and 6-fluoro-4-nitroisobenzofuran-1(3H)-one with acetic acid or acetic anhydride in the presence of water and a base to yield a compound of Formula II:

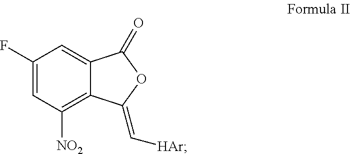

where HAr is as defined in the Summary of the Invention or as in any of the embodiments described herein.

In some or any embodiments, the method of preparing the compound of Formula I is according to General Scheme 1. In some or any embodiments, the compound of Formula I that is prepared is where the HAr is N-alkyl-1,2,4-triazolyl. In some or any embodiments, the alkyl is C₁₋₆ alkyl. In some or any embodiments, the alkyl is methyl, ethyl, or propyl. In some or any embodiments, the alkyl is propyl. In some or any embodiments, the compound of Formula I that is prepared is where the HAr is N-methyl-1,2,4-triazolyl or N-ethyl-1,2,4-triazolyl. In some or any embodiments, the compound of Formula I that is prepared is where the HAr is N-methyl-1,2,4-triazolyl. In some or any embodiments, the compound of Formula I that is prepared is where the HAr is N-alkyl-1,3,4-triazole. In some or any embodiments, the alkyl is C₁₋₆ alkyl. In some or any embodiments, the alkyl is methyl, ethyl, or propyl. In some or any embodiments, the alkyl is propyl. In some or any embodiments, the compound of Formula I that is prepared is where the HAr is N-methyl-1,3,4-triazolyl or N-ethyl-1,3,4-triazolyl. In some or any embodiments, the compound of Formula I that is prepared is where the HAr is N-methyl-1,3,4-triazolyl. In some or any embodiments, the compound of Formula I that is prepared is where the HAr is N-alkyl-1,2,3-triazole. In some or any embodiments, the alkyl is C₁₋₆ alkyl. In some or any embodiments, the alkyl is methyl, ethyl, or propyl. In some or any embodiments, the alkyl is propyl. In some or any embodiments, the compound of Formula I that is prepared is where the HAr is N-methyl-1,2,3-triazolyl or N-ethyl-1,2,3-triazolyl. In some or any embodiments, the compound of Formula I that is prepared is where the HAr is N-methyl-1,2,3-triazolyl.

In some or any embodiments, the method of preparing the Compound of Formula I is according to General Scheme 1 where the first solvent and the second solvent are the same. In certain embodiments, the first and second solvent are each independently selected from tetrahydrofuran, 2-methyl-tetrahydrofuran, an alkyl furan (a furan substituted with 1 or 2 C₁₋₄ alkyl groups), tert-butylmethylether, cyclopentylmethylether, or dioxane. In some or any embodiments, the method of preparing the Compound of Formula I is according to General Scheme 1 where the first solvent and the second solvent are 2-methyl-tetrahydrofuran. In some or any embodiments, the method of preparing the Compound of Formula I is according to General Scheme 1 where the first solvent and the second solvent are tetrahydrofuran.

In some or any embodiments, the method of preparing the Compound of Formula I is according to General Scheme 1 where the lithium base is LDA, LiNH₂, LiH, or LiHMDS. In certain embodiments, the lithium base is LiHMDS.

In some or any embodiments, the method of preparing the Compound of Formula I is according to General Scheme 1 where HAr-H (1) is treated with DMF and a lithium base in a first solvent to yield Compound I, wherein the lithium base is LDA, LiNH₂, LiH, or LiHMDS, and the first solvent is tetrahydrofuran, 2-methyl-tetrahydrofuran, a furan substituted with 1 or 2 C₁₋₄ alkyl groups, tert-butylmethylether, cyclopentylmethylether, or dioxane. In certain embodiments, the lithium base is LiHMDS, and the first solvent is tetrahydrofuran, 2-methyl-tetrahydrofuran, a furan substituted with 1 or 2 C₁₋₄ alkyl groups, tert-butylmethylether, cyclopentylmethylether, or dioxane. In certain embodiments, the lithium base is LDA, LiNH₂, LiH, or LiHMDS, and the first solvent is tetrahydrofuran or 2-methyl-tetrahydrofuran. In certain embodiments, the lithium base is LiHMDS, and the first solvent is 2-methyl-tetrahydrofuran.

In some or any embodiments, the method of preparing the Compound of Formula I is according to General Scheme 1 where Compound I precipitates as a solvate. In certain embodiments HAr-H (1) is treated with DMF and a lithium base in a first solvent to yield Compound I as a precipitated solvate. In certain embodiments, the first solvent is tetrahydrofuran or 2-methyl-tetrahydrofuran and the precipitate is a Compound I-tetrahydrofuran solvate or a compound I-2-methyl-tetrahydrofuran solvate. In certain embodiments, the first solvent is 2-methyl-tetrahydrofuran and the precipitate is a Compound I-2-methyl-tetrahydrofuran solvate.

In some or any embodiments, the method of preparing the Compound of Formula II is according to General Scheme 2, wherein the Compound of Formula I and 6-fluoro-4-nitroisobenzofuran-1(3H)-one (6) are treated with acetic acid or acetic anhydride in the presence of water and a base to yield a compound of Formula II. In some or any embodiments, the Compound of Formula II that is prepared is where the HAr is N-alkyl-1,2,4-triazolyl. In some or any embodiments, the alkyl is C₁₋₆ alkyl. In some or any embodiments, the alkyl is methyl, ethyl, or propyl. In some or any embodiments, the alkyl is methyl. In some or any embodiments, the alkyl is ethyl. In some or any embodiments, the alkyl is propyl. In some or any embodiments, the Compound of Formula II that is prepared is where the HAr is N-methyl-1,2,4-triazolyl or N-ethyl-1,2,4-triazolyl. In some or any embodiments, the Compound of Formula II that is prepared is where the HAr is N-methyl-1,2,4-triazolyl. In some or any embodiments, the Compound of Formula II that is prepared is where the HAr is N-alkyl-1,3,4-triazole. In some or any embodiments, the alkyl is C₁₋₆ alkyl. In some or any embodiments, the alkyl is methyl, ethyl, or propyl. In some or any embodiments, the alkyl is methyl. In some or any embodiments, the alkyl is ethyl. In some or any embodiments, the alkyl is propyl. In some or any embodiments, the Compound of Formula II that is prepared is where the HAr is N-methyl-1,3,4-triazolyl or N-ethyl-1,3,4-triazolyl. In some or any embodiments, the Compound of Formula II that is prepared is where the HAr is N-methyl-1,3,4-triazolyl. In some or any embodiments, the Compound of Formula II that is prepared is where the HAr is N-alkyl-1,2,3-triazole. In some or any embodiments, the alkyl is C₁₋₆ alkyl. In some or any embodiments, the alkyl is methyl, ethyl, or propyl. In some or any embodiments, the alkyl is methyl. In some or any embodiments, the alkyl is ethyl. In some or any embodiments, the alkyl is propyl. In some or any embodiments, the Compound of Formula II that is prepared is where the HAr is N-methyl-1,2,3-triazolyl or N-ethyl-1,2,3-triazolyl. In some or any embodiments, the Compound of Formula II that is prepared is where the HAr is N-methyl-1,2,3-triazolyl.

### Preparation of Compounds

The following are illustrative examples of how the compounds can be prepared and tested. Although the examples can represent only some embodiments, it should be understood that the following examples are illustrative and not limiting.

In a further aspect, it is provided a method of making a compound, comprising synthesizing a compound as any of the various embodiments described above or below. Examples of the method are further described in the Examples.

Compounds disclosed herein are commercially available or can be readily prepared from commercially available starting materials according to established methodology in the art of organic synthesis. General methods of synthesizing the compound can be found in, e.g., Stuart Warren and Paul Wyatt, Workbook for Organic Synthesis: The Disconnection Approach, second Edition, Wiley, 2010. Synthesis of some of the compounds are exemplified in detail below.

In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral axillary, separation of the resulting mixture of diastereomers by recrystallization or chromatography and liberation of the optically pure product from the auxiliary, or (2) direct separation of the mixture of optical enantiomers on chiral chromatographic column.

Materials were obtained from commercial suppliers and were used without further purification. Air or moisture sensitive reactions were conducted under argon or nitrogen atmosphere using oven-dried glassware and standard syringe/septa techniques. ¹H NMR and ¹³C-NMR spectra were measured at 400 MHz and 100 MHz, respectively, unless stated otherwise and data were reported as follows in ppm (6) from the internal standard (TMS, 0.0 ppm): chemical shift (multiplicity, integration, coupling constant in Hz).

### Synthetic Examples

### Example 1

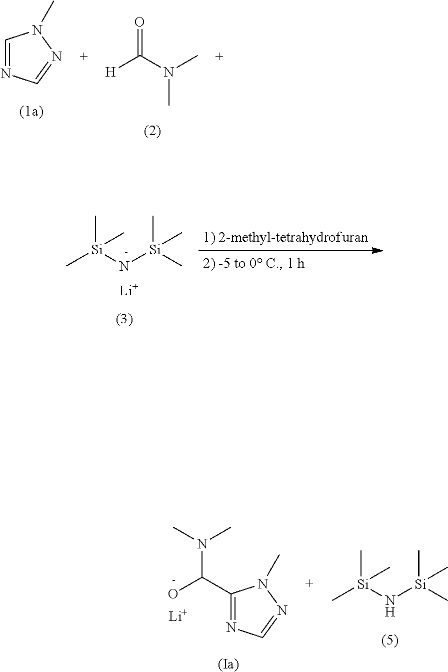

To a flask was added N-methyl-1,2,4-triazole (1a)(249.3 g, 3.0 mol, 1 equiv.), 2-methyl-THF (1020 mL, about 1:4 m/v), and DMF (2)(230.2 g, 3.15 mol, 1.05 equiv.), in any order. The solution was cooled to an internal temperature of about −5 to 0° C. To the flask was added LiHMDS (3) as a 20% solution in 2-methyl-THF (3012 g, 3.6 mol, 1.2 equiv.) dropwise within about 60 minutes. During the addition of the LiHMDS (3), the desired Compound (Ia) was precipitated as the 2-methyl-THF solvate, and the flask was cooled to about −30° C. The reaction was stirred for about 30 minutes at an internal temperature of about -5 to 0° C.

The precipitated crystals were removed from the reaction mixture by filtration and washed with 2-methyl-THF. The product, Compound (Ia) as the 2-methyl-THF solvate, was dried under vacuum at an internal temperature of about 60° C. (about 72.5% as measured by NMR) to yield Compound (Ia).

### Example 2

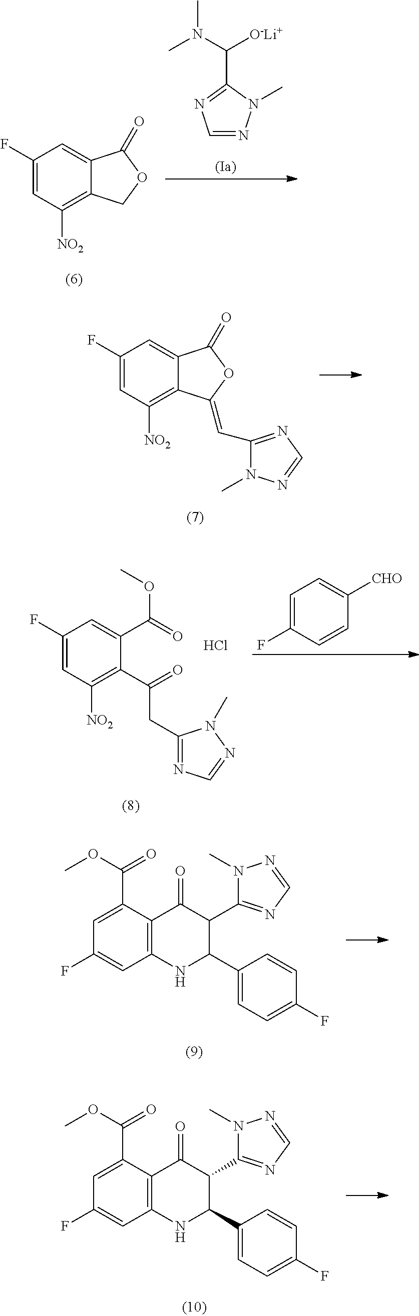

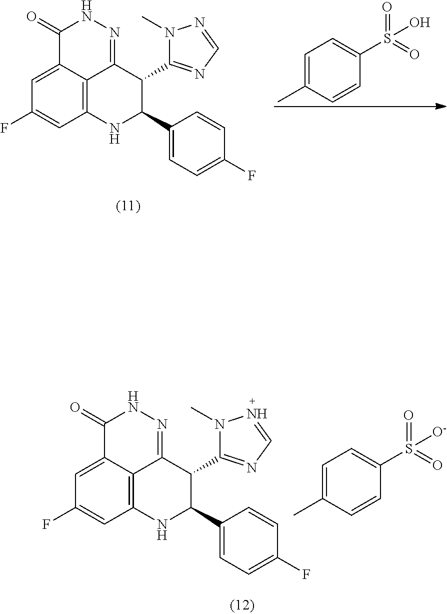 [SMILES: Cn1ncnc1[C@H]1c2n[nH]c(=O)c3cc(F)cc(c23)N[C@@H]1c1ccc(F)cc1 ; Cc1ccc(S(=O)(=O)O)cc1 ; Cn1ncnc1[C@H]1c2n[nH]c(=O)c3cc(F)cc(c23)N[C@@H]1c1ccc(F)cc1 ; Cc1ccc(S(=O)(=O)[O-])cc1]

As shown in Example 2, the Compounds of Formula I are useful in the synthesis of more complex compounds. See General Scheme 1 for a description of how the first step can be accomplished. Compounds of Formula I can be reacted with compound (6) to yield Compounds of Formula II. In Example 2, Compound (Ia) can be reacted with Compound (6) to yield Compound (7). The remaining steps are accomplished using procedures known to one of ordinary skill in the art, for example, as disclosed in WO2010017055 and WO2011097602 to yield Compound (12).

Other objects, features and advantages of the compounds, methods and compositions described herein will become apparent from the following description. It should be understood, however, that the description and the specific examples, while indicating specific embodiments, are given by way of illustration only, since various changes and modifications within the spirit and scope of the present description will become apparent from this detailed description.

All publications including patents, patent applications and published patent applications cited herein are hereby incorporated by reference for all purposes.

We claim:

1. A compound of Formula I: 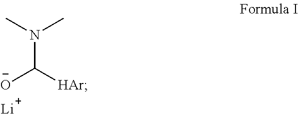
wherein HAr is N-alkyl-1,2,4-triazolyl, N-alkyl-1,3,4-triazolyl, or N-alkyl-1,2,3-triazolyl.

2. The compound of claim 1, wherein the alkyl is C₁₋₆ alkyl.

3. The compound of claim 1, wherein the HAr is N-alkyl-1,2,4-triazolyl.

4. The compound of claim 1, wherein the HAr is N-alkyl-1,3,4-triazolyl.

5. The compound of claim 1, wherein the HAr is N-alkyl-1,2,3-triazolyl.

6. The compound of any of claims 1-5, wherein the alkyl is methyl, ethyl, or propyl.

7. The compound of any of claims 1-6, wherein the alkyl is methyl.

8. A method of making a compound of Formula I according to any of claims 1-7, comprising treating a compound of formula HAr-H, or a salt thereof, with N,N-dimethylformamide in a first solvent followed by a lithium base; wherein HAr-H is N-alkyl-1,2,4-triazole, N-alkyl-1,3,4-triazole, or N-alkyl-1,2,3-triazole, and the lithium base is lithium diisopropylamide, lithium amide, lithium hydride, or lithium bis(trimethylsilyl)amide.

9. The method of claim 8, wherein the lithium base is lithium bis(trimethylsilyl)amide.

10. The method of claim 8, wherein the first solvent is tetrahydrofuran, 2-methyl-tetrahydrofuran, a furan substituted with 1 or 2 C₁₋₄ alkyl groups, tert-butylmethylether, cyclopentylmethylether, or dioxane.

11. The method of claim 8, wherein the first solvent is tetrahydrofuran or 2-methyl-tetrahydrofuran.

12. The method of claim 8, wherein the lithium base is lithium bis(trimethylsilyl)amide and the first solvent is 2-methyl-tetrahydrofuran.

13. The method of claim 12, wherein the compound of Formula I precipitates as a 2-methyl-tetrahydrofuran solvate.

14. The method of any of claims 8-13, wherein the alkyl is C₁₋₆ alkyl.

15. The method of any of claims 8-13, the HAr is N-alkyl-1,2,4-triazolyl.

16. The method of any of claims 8-13, wherein the HAr is N-alkyl-1,3,4-triazolyl.

17. The method of any of claims 8-13, wherein the HAr is N-alkyl-1,2,3-triazolyl.

18. The method of any of claims 8-13, wherein the alkyl is methyl, ethyl, or propyl.

19. The method of any of claims 8-13, wherein the alkyl is methyl.

20. The method of any of claims 8-19, further comprising treating the compound of Formula I and 6-fluoro-4-nitroisobenzofuran-1(3H)-one with acetic acid or acetic anhydride in the presence of water and a base to yield a compound of Formula II: 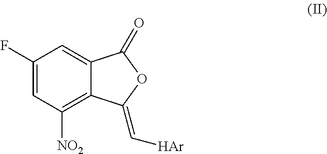
wherein HAr is N-alkyl-1,2,4-triazolyl, N-alkyl-1,3,4-triazolyl, or N-alkyl-1,2,3-triazolyl.

21. The method of claim 20, wherein the HAr is N-methyl-1,2,4-triazolyl.
